(19) United States
(12) Patent Application Publication
(10) Pub. No.: US 2026/0020885 A1
(43) Pub. Date: Jan. 22, 2026

(54) DEVICES AND METHODS FOR BONE FIXATION

(71) Applicant: DePuy Synthes Products, Inc., Raynham, MA (US)

(72) Inventor: Richard W. CASTLE, Honey Brook, PA (US)

(73) Assignee: DePuy Synthes Products, Inc., Raynham, MA (US)

(21) Appl. No.: 18/773,828

(22) Filed: Jul. 16, 2024

(51) Int. Cl. A61B 17/72; A61B 17/56

(52) U.S. Cl. CPC A61B 17/7258; A61B 17/725; A61B 2017/564

(57) ABSTRACT

An example apparatus is provided for the approximation of two bones. The apparatus can include a nail configured to be disposed within a first bone (e.g., the fibula). The nail can include a proximal end, a distal end, and a rigid body. The apparatus can further include one or more first threads disposed along the proximal end of the nail, and one or more second threads disposed along the distal end of the nail. The first thread(s) can be axially spaced from the second thread(s).

### FIELD

The present invention generally relates to devices and methods for bone fixation. More specifically, certain embodiments relate to systems and methods for fixation of the distal tibia and distal fibula following an injury to the corresponding syndesmotic joint.

### BACKGROUND

A syndesmotic injury results when a traumatic injury damages the ligaments that span the gap between the distal tibia and fibula. This can be the result of a high ankle sprain, with no fracture of the fibula, or can also accompany a fibular fracture in a Weber B or Weber C fracture.

A surgeon can determine the presence of a syndesmotic injury by direct visualization of the joint or through radiographic imaging while positioning the ankle in a mortise view orientation. In either case, loads are applied to the joint in either a direct lateral load applied to the fibula or by applying an external rotation load to the foot. While the load is being applied, the relative distance between the fibula and the tibia, the fibula and the talus, and the tibia and the talus are observed to determine the level of damage sustained by the ligaments that typically hold the syndesmotic joint together.

If a syndesmotic injury is found to be present, the typical treatment involves stabilizing the fibula and tibia with respect to each other in the proper orientation and holding them there throughout the soft tissue healing period to allow the ligaments to re-attach and heal. In the event of a syndesmotic injury with a corresponding fibula fracture, this is done while also stabilizing the fibular fracture, which is usually accomplished with a small fracture plate on the lateral side of the fibula. Traditionally the method of stabilization has been to place one or more cortical screws across the syndesmosis, with the head against the lateral face of the fibula and the tip of the screw being in the middle of the tibia or in the medial cortex of the tibia.

This form of treatment, whereby the lateral aspect of the syndesmotic fixation sits on the lateral face of the bone, can result in unstable ankle fractures as any weight or force provided by the lateral aspect can be transferred through the fibular fracture itself. Additionally, any retained hardware under a lateral malleolar incision can cause irritation and discomfort to the skin and soft tissue. Existing fibula nails attempt to address these concerns; however, they require complex insertion techniques and associated instrumentation.

Accordingly, alternative apparatus and methods for providing bone fixation, such as of the distal tibia and fibula following a syndesmotic injury, would be useful.

### SUMMARY

The present invention is directed to apparatus and methods for the approximation of two bones, e.g., the tibia and fibula.

In an exemplary application, the apparatus and methods herein can be configured to provide substantially rigid tensile fixation between the tibia and fibula while allowing the small amount of shear and rotational motion required for a standard gait. This can make it possible for patients to return to weight-bearing earlier, which can improve clinical outcomes, and/or can also reduce the number of follow-up hardware-removal surgeries.

An example apparatus is provided for the approximation of two bones. The apparatus can include a nail configured to be disposed within a first bone (e.g., the fibula). The nail can include a proximal end, a distal end, and a rigid body. The apparatus can further include one or more first threads disposed along the proximal end of the nail, and one or more second threads disposed along the distal end of the nail. The first thread(s) can be axially spaced from the second thread(s).

An example kit is provided for the approximation of two bones. The kit can include an apparatus which can include a nail and an insertion block. The nail can be configured to be delivered into a first bone (e.g., the fibula), and can include a proximal end, a distal end, first thread(s) disposed along the proximal end, second thread(s) disposed along the distal end, one or more fixation holes, and a distal tip. The insertion block can be configured to selectively engage with the distal tip of the nail. The kit can further include an arm configured to engage with the insertion block and includes one or more openings. The kit can further include a first installation tool configured to aid in delivering the nail into the first bone. The kit can further include a second installation tool configured to engage with the insertion block to aid in delivering the nail and the insertion block into the first bone. The kit can further include one or more sleeves configured to engage with the opening(s) of the arm to aid in delivering one or more fixation devices through the fixation hole(s) of the nail. The kit can further include a first removal tool configured to release the insertion block from the nail.

An example method is provided for the approximation of two bones. The method can include delivering an apparatus into a first bone. The apparatus can include a nail having a proximal end, a distal end, a distal tip, and a rigid body. The apparatus can further include first thread(s) disposed along the proximal end, second thread(s) disposed along the distal end, fixation hole(s) disposed between the first thread(s) and the second thread(s), and an insertion block engaged with the distal tip and extending distally beyond the first bone. The method can include engaging an arm with the insertion block via at least a locking knob of the arm, the arm having one or more openings. The method can include inserting a first sleeve through a first opening of the opening(s). The method can include drilling a first hole laterally through the first bone while using the first sleeve as a guide. The first hole can extend through a first fixation hole of the fixation hole(s). The method can include delivering a first fixation device into the first bone through the first hole and the first fixation hole. The method can include disengaging the insertion block from the distal tip of the nail thereby removing the insertion block, the arm, the locking knob, and the first sleeve from the nail and leaving the nail in the first bone.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIG. 1 is an illustration of an example system for bone fixation, according to aspects of the present invention.

FIG. 2 is a perspective view of an example apparatus used for the approximation of two bones, according to aspects of the present invention.

FIG. 3 is an illustration of an example kit used for the approximation of two bones, according to aspects of the present invention.

FIGS. 4A-4B illustrate steps of a method for the approximation of two bones, according to aspects of the present invention.

FIGS. 5A-5B illustrate steps of a method for the approximation of two bones, according to aspects of the present invention.

FIG. 6 illustrates a step of a method for the approximation of two bones, according to aspects of the present invention.

FIG. 7 illustrates a step of a method for the approximation of two bones, according to aspects of the present invention.

FIG. 8 illustrates a cross-sectional view of various components used in a step of a method for the approximation of two bones, according to aspects of the present invention.

FIG. 9 illustrates a step of a method for the approximation of two bones, according to aspects of the present invention.

FIG. 10A is an illustration of an example nail for the approximation of two bones, according to aspects of the present invention.

FIG. 10B is an illustration of an example nail for the approximation of two bones, according to aspects of the present invention.

FIG. 10C is an illustration of an example nail for the approximation of two bones, according to aspects of the present invention.

FIG. 11 is a flowchart of a method for the approximation of two bones, according to aspects of the present invention.

### DETAILED DESCRIPTION

As used herein, the terms “about” or “approximately” for any numerical values or ranges indicate a suitable dimensional tolerance that allows the part or collection of components to function for its intended purpose as described herein. More specifically, “about” or “approximately” can refer to the range of values±20% of the recited value, e.g., “about 50” can refer to the range of values from 40 to 60.

The example devices and methods of treatment described herein generally involve providing approximation (e.g., syndesmosis repair) of two bones, such as the tibia and fibula bones, via lateral fixation in the fibula bone, and/or distal fixation of a support structure (e.g., a nail) in a bone, such as the fibula bone. That is, a support, such as a nail, can be implanted in the fibula bone such that one or more fixation devices (e.g., screws) can be delivered through the nail for the approximation of the fibula and tibia. This configuration can help to prevent an unstable fibular fracture, and/or decrease patient recovery time associated with a fibular fracture, as any weight or force placed on the syndesmotic injury during recovery (e.g., via movement) can be transferred through the nail as opposed to the fibular fracture itself.

Various example systems and methods are presented herein. Features from each example are combinable with other examples as understood by persons skilled in the pertinent art.

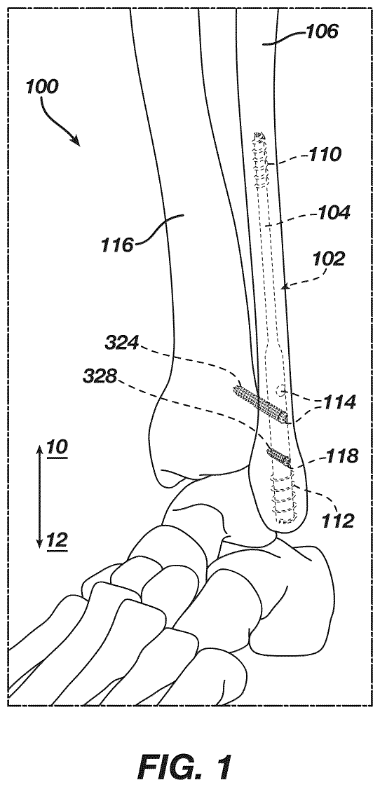

FIG. 1 is an example system 100 for the approximation of a first bone 106 (e.g., the fibula) and a second bone 116 (e.g., the tibia). As further discussed below, an apparatus 102 can be delivered proximally 10 into the first bone 106 such that the apparatus 102 can aid in the approximation of the first and second bones 106, 116.

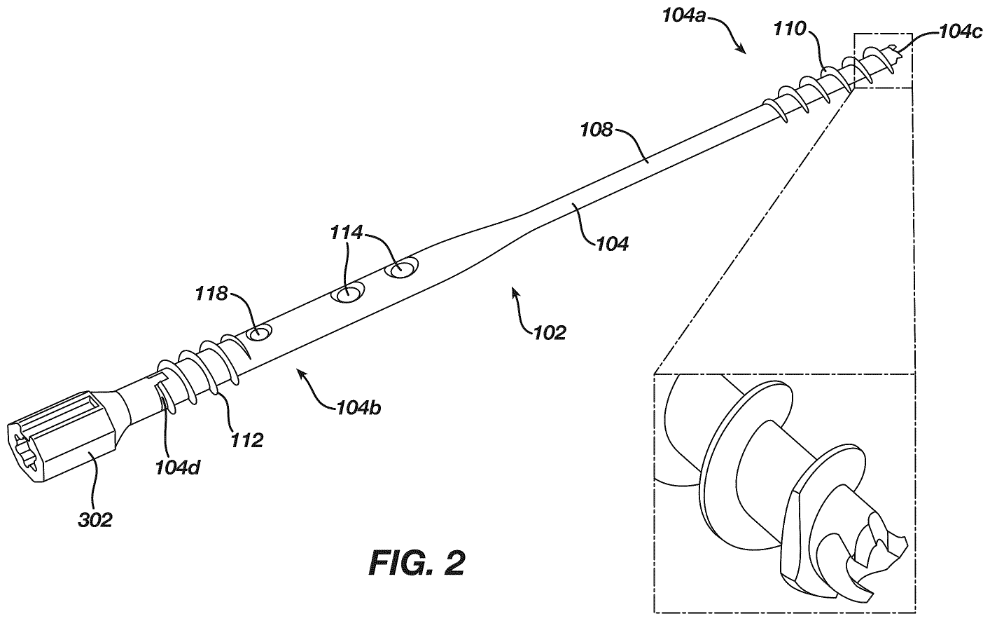

FIG. 2 provides a perspective view of the apparatus 102. As shown, the apparatus 102 can include a nail 104, which itself can include a proximal end 104a, a distal end 104b, and a rigid body 108. In some embodiments, the rigid body 108 can be a monolithic structure. The apparatus 102 can further include first thread(s) 110 disposed along the proximal end 104a of the nail 104, and second thread(s) 112 disposed along the distal end 104b of the nail 104.

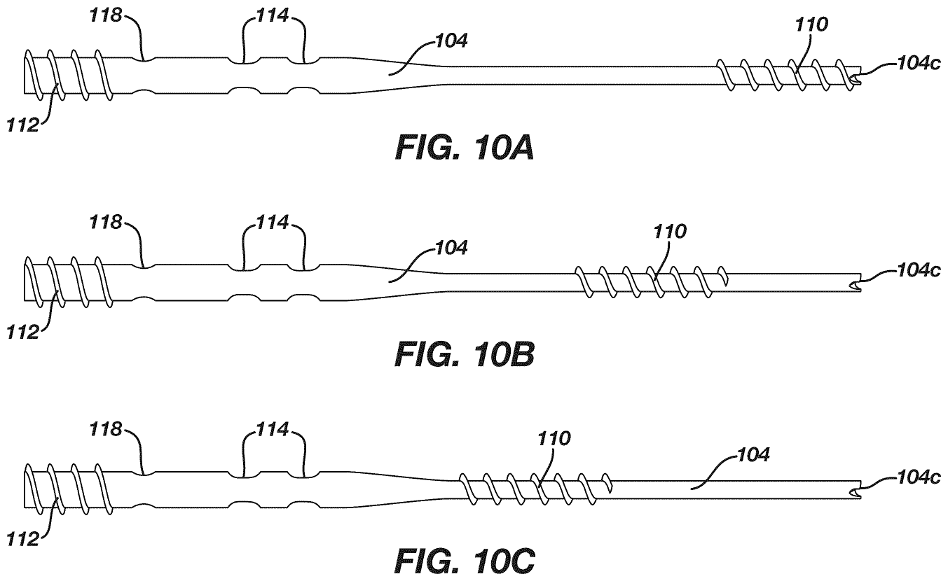

In some embodiments, the apparatus 102 can include a proximal cutting tip 104c of the nail 104. As further discussed below, the proximal cutting tip 104c can aid in delivering the nail 104 into the first bone 106. In some embodiments, the first thread(s) 110 can be axially spaced from the second thread(s) 112. As particularly shown in FIGS. 10A-10C, the first thread(s) 110 can be disposed adjacent to the proximal cutting tip 104c (FIG. 10A), or disposed along the proximal end 104a of the nail 104 such that there is a predetermined amount of space between the first thread(s) 110 and the proximal cutting tip 104c (FIGS. 10B-10C).

Turning back to FIG. 2, the apparatus 102 can further include one or more first fixation holes 114 in the nail 104. The first fixation hole(s) 114 can aid in fixating the first bone 106 to the second bone 116 via one or more fixation devices (e.g., screws), as particularly shown in FIG. 1. The first fixation hole(s) 114 can be disposed between the first thread(s) 110 and the second thread(s) 112. The apparatus 102 can further include one or more second fixation holes 118 in the nail 104. The second fixation hole(s) 118 can aid in fixating the nail 104 to the first bone 106. That is, the second fixation hole(s) 118 can help to lock the nail 104 in place after it has been delivered into the first bone 106, as further discussed below. The second fixation hole(s) 118 can be disposed between the first and second thread(s) 110, 112.

In some embodiments, the apparatus 102 can include an insertion block 302 removably attached to the distal end 104b of the nail 104. Specifically, the insertion block 302 can engage with a distal tip 104d of the nail 104. As further discussed below, the insertion block 302 can aid in delivering the apparatus 102 into the first bone 106, can extend distally 12 beyond the first bone 106 when the apparatus 102 is delivered into the first bone 106, and/or can be disengaged from the nail 104 once the nail 104 is delivered into the first bone 106.

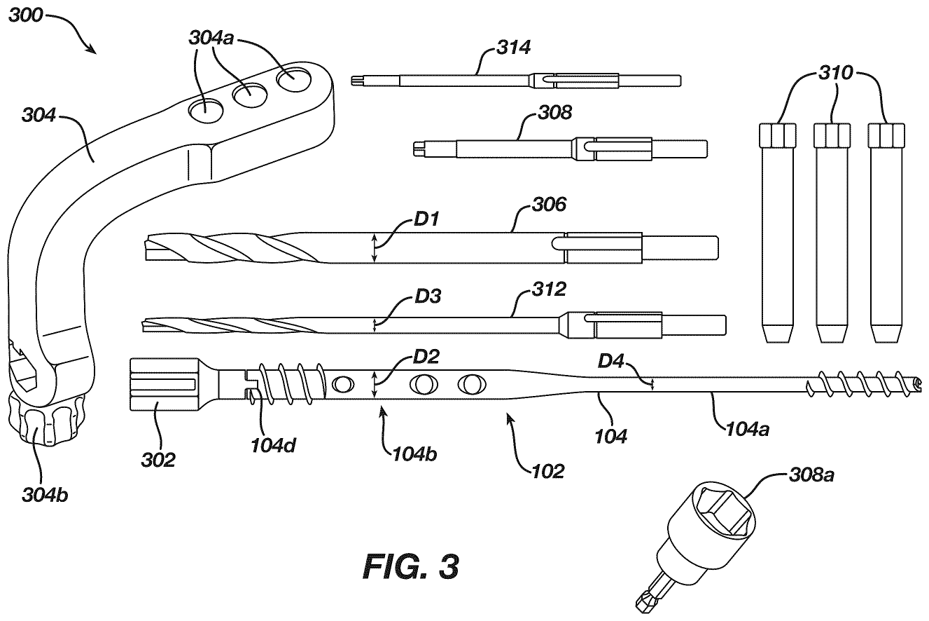

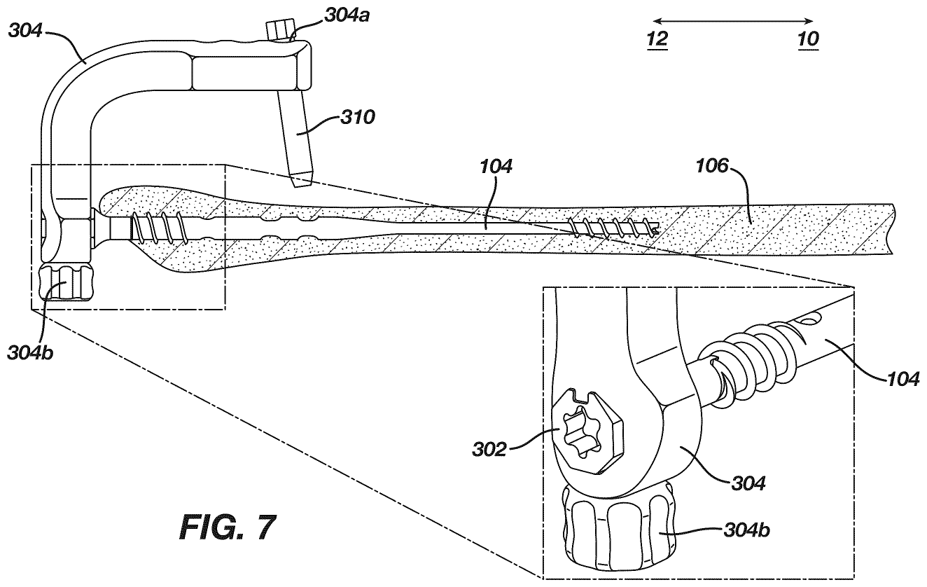

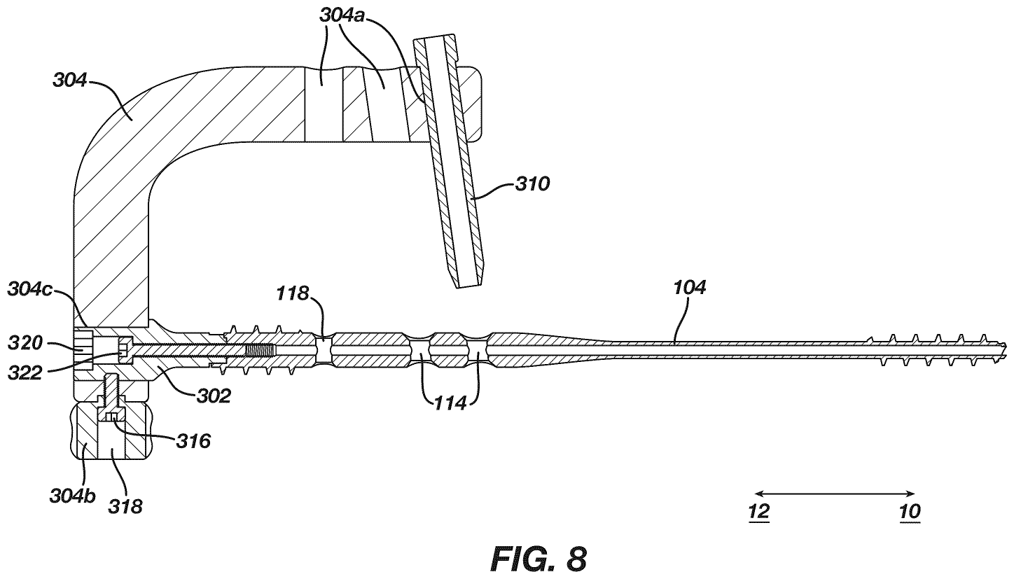

FIG. 3 provides an illustration of a kit assembly 300 that can be used in the approximation of two bones. The kit 300 can include an apparatus 102, as described herein. The kit 300 can include an arm 304 configured to engage with the insertion block 302, e.g., as shown and described below with respect to FIGS. 7 and 8. The arm 304 can include one or more openings 304a that can aid in approximating the first and second bones 106, 116 and/or locking the nail 104 in place within the first bone 106, as discussed herein. The arm 304 can also include a locking knob 304b configured to engage with the insertion block 302, as further discussed below.

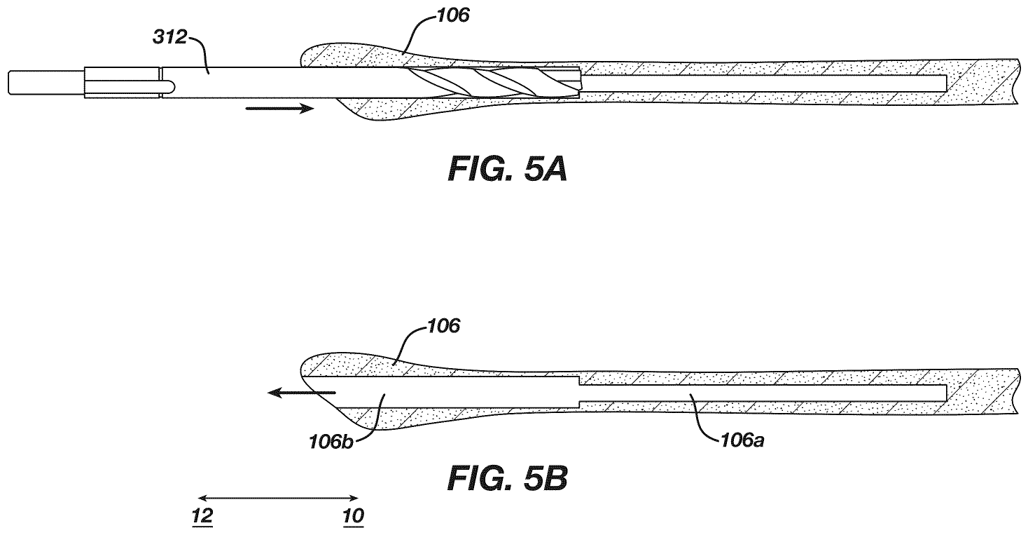

The kit 300 can include a first installation tool 306 that can be configured to aid in delivering the nail 104 into the first bone. In some embodiments, the first installation tool 306 can have a diameter D1 sized for accommodating a diameter D2 of the distal end 104b of the nail 104, as further discussed below with respect to FIGS. 5A-5B. In some embodiments, the fixation hole(s) 114, 118 of the nail 104, as discussed herein, can be disposed along a section of the nail 104 that has the diameter D2.

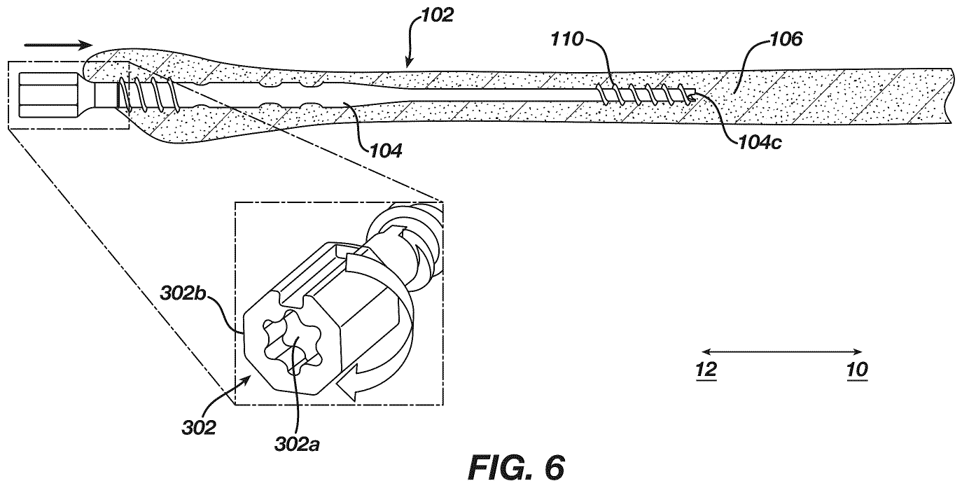

The kit 300 can include a second installation tool 308 that can be configured to engage with the insertion block 302 to aid in delivering the nail 104 and the insertion block 302 into the first bone 106. In some embodiments, rather than use second installation tool 308 (e.g., a star drive), a user can instead use second installation tool 308a (e.g., an outer hex) to engage with the insertion block 302, as further discussed below with respect to FIG. 6.

The kit 300 can include one or more sleeves 310 configured to engage with the opening(s) 304a of the arm 304 to aid in delivering one or more fixation devices (e.g., screws) through the fixation hole(s) 114, 118 of the nail 104, as further discussed below with respect to FIGS. 7-9.

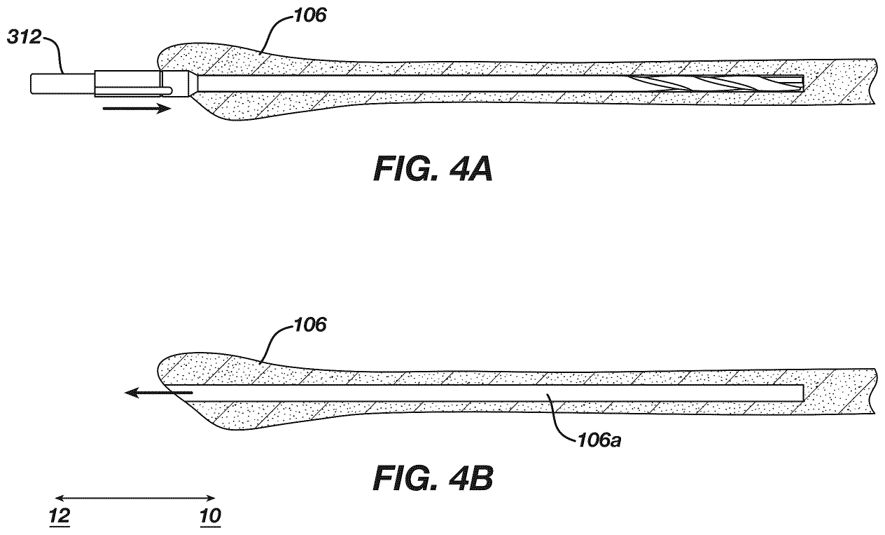

The kit 300 can include a third installation tool 312 configured to aid in delivering the nail 104 into the first bone. The third installation tool 312 can have a diameter D3 sized for accommodating a diameter D4 of the proximal end 104a of the nail 104, as further discussed below with respect to FIGS. 4A-4B.

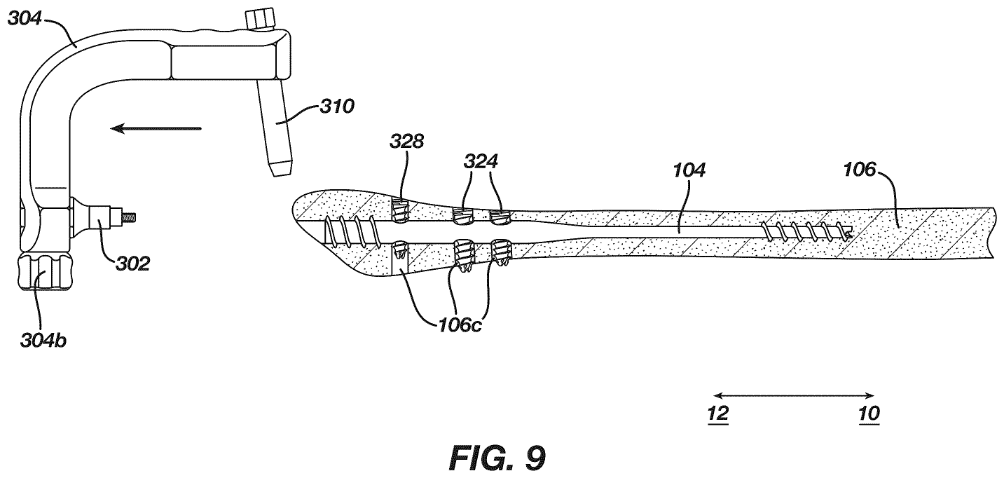

The kit 300 can include a first removal tool 314 configured to release the insertion block 302 from the nail 104, such as after the nail 104 is delivered into the first bone 106, as further discussed below with respect to FIG. 9.

FIGS. 4A-4B illustrate steps of a method for the approximation of two bones. In some embodiments, the method can include drilling a hole proximally through the first bone 106 using the third installation tool 312 (FIG. 4A) to create a first channel 106a (FIG. 4B) in the first bone 106. As discussed above, the third installation tool 312 can have a diameter D3 sized for accommodating the diameter D4 of the proximal end 104a of the nail 104. As such, a user can drill this first hole using the third installation tool 312 to be able to later deliver the nail 104 into the first bone 106, as further discussed below. Alternatively, rather than use third installation tool 312, the proximal cutting tip 104c and/or the first thread(s) 110 of the nail 104 can be used to bore the first channel 106a into the first bone 106. For example, a user can engage a second installation tool 308 or 308a with the insertion block 302, as further discussed below, to screw the apparatus 102 into the first bone 106, thereby boring the first channel 106a into the first bone 106 using the proximal cutting tip 104c and/or the first thread(s) 110 of the nail 104.

As shown in FIGS. 5A-5B, the method can include drilling a hole proximally through the first bone 106 using the first installation tool 306 (FIG. 5A) to create a second channel 106b (FIG. 5B) in the first bone 106. As discussed above, the first installation tool 306 can have a diameter D1 sized for accommodating the diameter D2 of the distal end 104b of the nail 104. The diameter D4 of the proximal end 104a of the nail 104 can be smaller than the diameter D2 of the distal end 104b of the nail 104. As such, when the apparatus 102 is delivered proximally into the first bone 106, as further discussed below, the proximal end 104a of the nail 104 can fit into the first channel 106a, with the second channel 106b being larger to accommodate the larger diameter of the distal end 104b of the nail 104.

As shown in FIG. 6, the method can include delivering the apparatus 102 into the first bone 106. As discussed above, a user can engage either second installation tool 308 or 308a (FIG. 3) with the insertion block 302. For example, second installation tool 308 can be a star drive that can engage with an inner recess 302a (e.g., a star drive recess) of the insertion block 302. As another example, second installation tool 308a can be an outer hex that can engage with an outer edge 302b of the insertion block 302. In either scenario, once engaged with the insertion block 302, either the second installation tool 308 or 308a can be rotated thereby actuating or rotating the insertion block 302 and consequently the nail 104 to aid in delivering the apparatus 102 proximally into the first bone 106.

As shown in FIGS. 7 and 8, the method can include engaging an arm 304 with the insertion block 302. As particularly shown in FIG. 8, the arm 304 can be engaged with the insertion block 302 by sliding the insertion block 302 into a channel 304c of the arm 304 and/or by engaging a locking knob 304b of the arm 304 with the insertion block 302 using a screw or other fixation device 316. The fixation device 316 can be delivered through an internal channel 318 of the locking knob 304b, and upwards through the arm 304 and/or the insertion block 302 to lock the components in place with respect to one another.

As further shown in FIGS. 7 and 8, the method can include inserting a first sleeve 310 through a first opening of the opening(s) 304a of the arm 304. The sleeve 310 can help a user (e.g., a surgeon) drill a hole 106c (FIG. 9) laterally through the first bone 106, wherein the first hole 106c can extend through one of the fixation hole(s) 114, 118. The user can then deliver a fixation device (e.g., a screw) into the first bone 106 through the hole 106c and the applicable fixation hole 114, 118. For example, the user can use the sleeve 310 as a guide for properly aligning a drill with the first bone 106 such that the user can drill a hole 106c (FIG. 9) through the first bone 106 and the applicable fixation hole 114, 118. This process can be repeated such that a sleeve 310 can be inserted into additional opening(s) 304a of the arm 304 to help drill properly aligned hole(s) 106c to fixate the nail 104 to the first bone 106 and/or fixate the first and second bones 106, 116 to one another. For example, a hole 106c can be drilled through one or more of the first fixation hole(s) 114 of the nail 104, and a fixation device 324 delivered through the hole 106c and the first fixation hole 114. As discussed herein (and shown in FIG. 1), first fixation device 324 can be further inserted into second bone 116 to aid in approximating the first and second bones 106, 116 (e.g., syndesmotic fixation). As another example, a hole 106c can be drilled through a second fixation hole 118 of the nail 104, and a fixation device 328 delivered through the hole 106c and the second fixation hole 118. Second fixation hole 118 can aid in fixating or locking the nail 104 to the first bone 106 for additional structural stability.

In some embodiments, the sleeve 310 can include one or more nested components, for example, an outer soft tissue sleeve, a drill sleeve, and an inner trocar. An outer soft tissue sleeve can assist a user (e.g., a surgeon) in properly aligning the sleeve so as to not damage surrounding soft tissue; the drill sleeve can assist in inserting a drill down through the outer sleeve; and the trocar can assist in making a necessary puncture and/or incision in the outer tissue layers.

As shown in FIG. 9, once the apparatus 102 is delivered into the first bone 106, the method can include disengaging the insertion block 302 from the distal tip 104d of the nail 104, thereby removing the insertion block 302, the arm 304, the locking knob 304b, and the sleeve(s) 310 from the nail 104, leaving the nail 104 in the first bone 106. In some embodiments, the insertion block 302 can be disengaged from the distal tip 104d of the nail 104 by using a first removal tool 314 (FIG. 3). First removal tool 314 can be inserted into internal channel 320 of the insertion block 302 to disengage (e.g., unscrew) a fixation device 322 engaged with the distal tip 104d of the nail 104, as particularly shown in FIG. 8. The first removal tool 314 can have a smaller diameter compared to second installation tool 308 such that first removal tool 314 can fit within channel 320 of the insertion block 302.

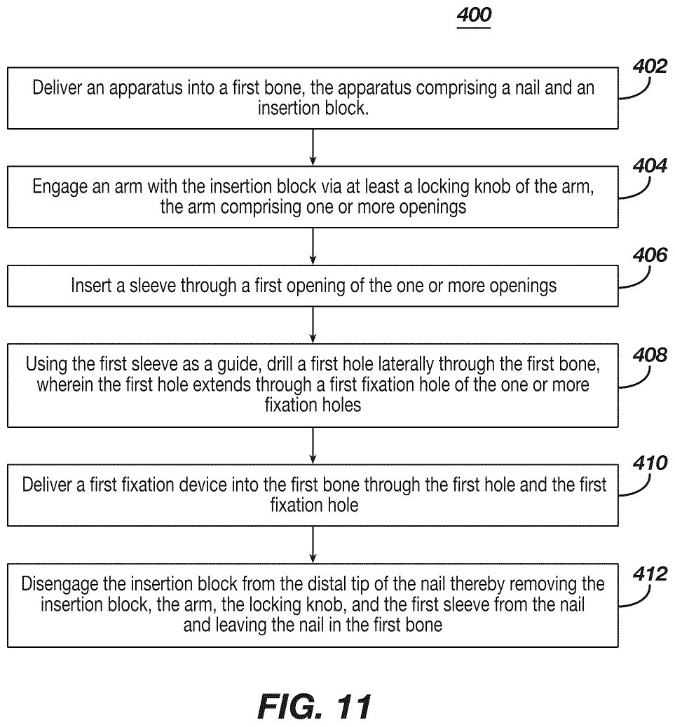

FIG. 11 provides a flowchart of an example method 400 for the approximation of two bones. The steps of method 400 can be performed by one or more devices or components discussed herein (e.g., apparatus 102, kit 300, etc.). It should be understood that certain embodiments of the disclosed technology can omit one or more blocks as being optional.

In block 402, the method can include delivering an apparatus (e.g., apparatus 102) into a first bone (e.g., first bone 106). This step can be the same as or similar to that described above with respect to FIGS. 4A-4B, 5A-5B, and 6.

In block 404, the method can include engaging an arm with the insertion block via at least a locking knob of the arm, the arm comprising one or more openings. This step can be the same as or similar to that described above with respect to FIGS. 7-8.

In block 406, the method can include inserting a first sleeve through a first opening of the one or more openings of the arm. This step can be the same as or similar to that described above with respect to FIGS. 7-8.

In block 408, the method can include drilling a first hole laterally through the first bone while using the first sleeve as a guide, the first hole extending through a first fixation hole of the one or more fixation holes. This step can be the same as or similar to that described above with respect to FIGS. 7-9.

In block 410, the method can include delivering a first fixation device into the first bone through the first hole and the first fixation hole. This step can be the same as or similar to that described above with respect to FIGS. 7-9.

In block 412, the method can include disengaging the insertion block from the distal tip of the nail thereby removing the insertion block, the arm, the locking knob, and the first sleeve from the nail and leaving the nail in the first bone. This step can be the same as or similar to that described above with respect to FIG. 9.

The descriptions contained herein are examples of embodiments of the invention and are not intended in any way to limit the scope of the invention. As described herein, the invention contemplates many variations and modifications of structures and methods, including alternative materials, alternative configurations of component parts, and alternative method steps. Modifications and variations apparent to those having skill in the pertinent art according to the teachings of this disclosure are intended to be within the scope of the claims which follow.

The disclosed technology described herein can be further understood according to the following clauses.

Clause 1: An apparatus for the approximation of two bones, comprising: a nail configured to be disposed within a first bone, the nail comprising a proximal end, a distal end, and a rigid body; one or more first threads disposed along the proximal end of the nail; and one or more second threads disposed along the distal end of the nail, the one or more first threads being axially spaced from the one or more second threads.

Clause 2: The apparatus of clause 1, further comprising: a proximal cutting tip disposed at the proximal end of the nail.

Clause 3: The apparatus of clause 2, wherein the one or more first threads are disposed adjacent to the proximal cutting tip.

Clause 4: The apparatus of clause 2, wherein the one or more first threads are disposed along the proximal end of the nail such that there is space between the one or more first threads and the proximal cutting tip.

Clause 5: The apparatus of clause 1, further comprising: one or more first fixation holes in the nail for fixating the first bone to a second bone via one or more first fixation devices, the one or more first fixation holes disposed between the one or more first threads and the one or more second threads.

Clause 6: The apparatus of clause 5, further comprising: one or more second fixation holes in the nail for fixating the nail to the first bone via one or more second fixation devices, the one or more second fixation holes disposed between the one or more first threads and the one or more second threads.

Clause 7: The apparatus of clause 6, wherein the one or more first and second fixation devices comprise screws.

Clause 8: The apparatus of clause 1, further comprising: an insertion block removably attached to the distal end of the nail.

Clause 9: A kit for the approximation of two bones, the kit comprising: an apparatus comprising: a nail configured to be delivered into a first bone and comprising: a proximal end; a distal end; one or more first threads disposed along the proximal end; one or more second threads disposed along the distal end; one or more fixation holes; and a distal tip; and an insertion block configured to selectively engage with the distal tip of the nail; an arm configured to engage with the insertion block and comprising one or more openings; a first installation tool configured to aid in delivering the nail into the first bone; a second installation tool configured to engage with the insertion block 302 to aid in delivering the nail and the insertion block into the first bone; one or more sleeves configured to engage with the one or more openings of the arm to aid in delivering one or more fixation devices through the one or more fixation holes of the nail; and a first removal tool configured to release the insertion block from the nail.

Clause 10: The kit of clause 9, wherein the first installation tool comprises a first diameter sized for accommodating a second diameter of the distal end of the nail.

Clause 11: The kit of clause 10, wherein the one or more fixation holes are disposed along a first section of the distal end of the nail, the first section comprising the second diameter.

Clause 12: The kit of clause 10, further comprising: a third installation tool comprising a third diameter sized for accommodating a fourth diameter of the proximal end of the nail.

Clause 13: The kit of clause 9, wherein the first removal tool releases the insertion block from the nail after the nail is delivered into the first bone.

Clause 14: The kit of clause 9, wherein the arm further comprises a locking knob configured to engage with the insertion block.

Clause 15: The kit of clause 14, wherein the insertion block, the arm, and the locking knob are removed from the nail after the nail is delivered into the first bone.

Clause 16: A method for the approximation of two bones, the method comprising: delivering an apparatus into a first bone, the apparatus comprising: a nail comprising a proximal end, a distal end, a distal tip, and a rigid body; one or more first threads disposed along the proximal end; one or more second threads disposed along the distal end; one or more fixation holes disposed between the one or more first threads and the one or more second threads; and an insertion block engaged with the distal tip and extending distally beyond the first bone; engaging an arm with the insertion block via at least a locking knob of the arm, the arm comprising one or more openings; inserting a first sleeve through a first opening of the one or more openings; using the first sleeve as a guide, drilling a first hole laterally through the first bone, wherein the first hole extends through a first fixation hole of the one or more fixation holes; delivering a first fixation device into the first bone through the first hole and the first fixation hole; and disengaging the insertion block from the distal tip of the nail thereby removing the insertion block, the arm, the locking knob, and the first sleeve from the nail and leaving the nail in the first bone.

Clause 17: The method of clause 16, wherein delivering the apparatus into the first bone comprises: drilling a first channel into the first bone using a first installation tool; drilling a second channel into the first bone using a second installation tool; and inserting the apparatus proximally into the first and second channels.

Clause 18: The method of clause 17, wherein: the first installation tool comprises a first diameter sized for accommodating a second diameter of the proximal end of the nail; and the second installation tool comprises a third diameter sized for accommodating a fourth diameter of the distal end of the nail.

Clause 19: The method of clause 18, wherein the second diameter is smaller than the fourth diameter.

Clause 20: The method of clause 16, wherein the nail further comprises a proximal cutting tip, and wherein delivering the apparatus into the first bone comprises: drilling a first channel into the first bone using a first installation tool; inserting the proximal end of the nail proximally into the first channel; and actuating the insertion block thereby boring a second channel into the first bone via one or more of the proximal cutting tip and the one or more first threads of the nail.

Clause 21: The method of clause 20, wherein actuating the insertion block comprises rotating the insertion block thereby rotating the nail.

Clause 22: The method of clause 16, further comprising: inserting a second sleeve through a second opening of the one or more openings; using the second sleeve as a guide, drilling a second hole laterally through the first bone, wherein the second hole extends through a second fixation hole of the one or more fixation holes; and delivering a second fixation device into the first bone through the second hole and the second fixation hole, wherein the first fixation device fixates the first bone to a second bone, and wherein the second fixation device fixates the nail to the first bone.

1. An apparatus for the approximation of two bones, comprising:
a nail configured to be disposed within a first bone, the nail comprising a proximal end, a distal end, and a rigid body;
one or more first threads disposed along the proximal end of the nail; and
one or more second threads disposed along the distal end of the nail, the one or more first threads being axially spaced from the one or more second threads.

2. The apparatus of claim 1, further comprising:
a proximal cutting tip disposed at the proximal end of the nail.

3. The apparatus of claim 2, wherein the one or more first threads are disposed adjacent to the proximal cutting tip.

4. The apparatus of claim 2, wherein the one or more first threads are disposed along the proximal end of the nail such that there is space between the one or more first threads and the proximal cutting tip.

5. The apparatus of claim 1, further comprising:
one or more first fixation holes in the nail for fixating the first bone to a second bone via one or more first fixation devices, the one or more first fixation holes disposed between the one or more first threads and the one or more second threads.

6. The apparatus of claim 5, further comprising:
one or more second fixation holes in the nail for fixating the nail to the first bone via one or more second fixation devices, the one or more second fixation holes disposed between the one or more first threads and the one or more second threads.

7. The apparatus of claim 1, further comprising:
an insertion block removably attached to the distal end of the nail.

8-20. (canceled)

21. A kit for the approximation of two bones, the kit comprising:
the apparatus of claim 7;
an arm configured to engage with the insertion block and comprising one or more openings;
a first installation tool configured to aid in delivering the nail into the first bone;
a second installation tool configured to engage with the insertion block to aid in delivering the nail and the insertion block into the first bone;
one or more sleeves configured to engage with the one or more openings of the arm to aid in delivering one or more fixation devices through one or more fixation holes of the nail; and
a first removal tool configured to release the insertion block from the nail.

22. The kit of claim 21, wherein the first installation tool comprises a first diameter sized for accommodating a second diameter of the distal end of the nail.

23. The kit of claim 22, wherein the one or more fixation holes are disposed along a first section of the distal end of the nail, the first section comprising the second diameter.

24. The kit of claim 21, further comprising:
a third installation tool comprising a third diameter sized for accommodating a fourth diameter of the proximal end of the nail.

25. The kit of claim 21, wherein the first removal tool releases the insertion block from the nail after the nail is delivered into the first bone.

26. The kit of claim 21, wherein:
the arm further comprises a locking knob configured to engage with the insertion block, and
the insertion block, the arm, and the locking knob are removed from the nail after the nail is delivered into the first bone.

27. A method for the approximation of two bones, the method comprising:
delivering the apparatus of claim 7 into a first bone;
engaging an arm with the insertion block via at least a locking knob of the arm, the arm comprising one or more openings;
inserting a first sleeve through a first opening of the one or more openings;
using the first sleeve as a guide, drilling a first hole laterally through the first bone, wherein the first hole extends through a first fixation hole of the one or more fixation holes;
delivering a first fixation device into the first bone through the first hole and the first fixation hole; and
disengaging the insertion block from the distal end of the nail thereby removing the insertion block, the arm, the locking knob, and the first sleeve from the nail and leaving the nail in the first bone.

28. The method of claim 27, wherein delivering the apparatus into the first bone comprises:
drilling a first channel into the first bone using a first installation tool;
drilling a second channel into the first bone using a second installation tool; and
inserting the apparatus proximally into the first and second channels.

29. The method of claim 28, wherein:
the first installation tool comprises a first diameter sized for accommodating a second diameter of the proximal end of the nail; and
the second installation tool comprises a third diameter sized for accommodating a fourth diameter of the distal end of the nail.

30. The method of claim 29, wherein the second diameter is smaller than the fourth diameter.

31. The method of claim 27, wherein the nail further comprises a proximal cutting tip, and wherein delivering the apparatus into the first bone comprises:
drilling a first channel into the first bone using a first installation tool;
inserting the proximal end of the nail proximally into the first channel; and
actuating the insertion block thereby boring a second channel into the first bone via one or more of the proximal cutting tip and the one or more first threads of the nail.

32. The method of claim 31, wherein actuating the insertion block comprises rotating the insertion block thereby rotating the nail.

33. The method of claim 27, further comprising:
inserting a second sleeve through a second opening of the one or more openings;
using the second sleeve as a guide, drilling a second hole laterally through the first bone, wherein the second hole extends through a second fixation hole of the one or more fixation holes; and
delivering a second fixation device into the first bone through the second hole and the second fixation hole,
wherein the first fixation device fixates the first bone to a second bone, and
wherein the second fixation device fixates the nail to the first bone.
